(19) United States
(12) Patent Application Publication
(10) Pub. No.: US 2008/0208308 A1
(43) Pub. Date: Aug. 28, 2008

(54) High Temperature Oxidation-Reduction Process to Form Porous Structures on a Medical Implant

(75) Inventors: Jeffrey Allen, Santa Rosa, CA (US); Matthew Bridsall, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/679,229

(22) Filed: Feb. 27, 2007

(51) Int. Cl. A61F 2/06

(52) U.S. Cl. 623/1.11; 623/1.15

(57) ABSTRACT

A system for treating abnormalities of the cardiovascular system includes a stent having a porous therapeutic agent carrying zone comprising oxidation and reduction products of one or more metals in the stent framework. Another embodiment of the invention includes a method of manufacturing a therapeutic agent carrying stent comprising exposing a metallic stent framework to oxidizing and reducting conditions, and forming a therapeutic agent carrying zone on the surface of the stent framework that includes oxidation and reduction products of one or more metals in the stent framework.

### TECHNICAL FIELD

This invention relates generally to biomedical devices that are used for treating vascular conditions. More specifically, the invention relates to a therapeutic agent eluting stent having one or more therapeutic agent eluting structures.

### BACKGROUND OF THE INVENTION

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion into the body, the stent is positioned in a compressed configuration on the delivery device. For example, the stent may be crimped onto a balloon that is folded or otherwise wrapped about the distal portion of a catheter body that is part of the delivery device. After the stent is positioned across the lesion, it is expanded by the delivery device, causing the diameter of the stent to expand. For a self-expanding stent, commonly a sheath is retracted, allowing the stent to expand.

Stents are used in conjunction with balloon catheters in a variety of medical therapeutic applications, including intravascular angioplasty to treat a lesion such as plaque or thrombus. For example, a balloon catheter device is inflated during percutaneous transluminal coronary angioplasty (PTCA) to dilate a stenotic blood vessel. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To reduce restenosis, stents, constructed of metals or polymers, are implanted within the vessel to maintain lumen size. The stent is sufficiently longitudinally flexible so that it can be transported through the cardiovascular system. In addition, the stent requires sufficient radial strength to enable it to act as a scaffold and support the lumen wall in a circular, open configuration. Configurations of stents include a helical coil, and a cylindrical sleeve defined by a mesh, which may be supported by a stent framework of struts or a series of rings fastened together by linear connector portions.

Stent insertion may cause undesirable reactions such as inflammation resulting from a foreign body reaction, infection, thrombosis, and proliferation of cell growth that occludes the blood vessel. Stents capable of delivering one or more therapeutic agents have been used to treat the damaged vessel and reduce the incidence of deleterious conditions including thrombosis and restenosis.

Polymer coatings applied to the surface of the stents have been used to deliver drugs or other therapeutic agents at the placement site of the stent. The coating is sometimes damaged during expansion of the stent at the delivery site, causing the coating to chip off the stent and release flakes of the polymer coating, which reduces the effective dose of the drug at the treatment site, and under some circumstances, may result in emboli in the microvasculature.

Recently, stents have been introduced that have a porous, nonpolymeric coating on the surface of the stent comprising a continuous metal oxide zone. A zone of, for example, aluminum oxide, magnesium oxide or titanium oxide is formed electrolytically on the surface of the stent framework. The size of the pores in the metal oxide zone can be modified by an appropriate adjustment of the applied voltage during metal oxide formation. In other processes, a continuos metal oxide zone is formed by heating the metallic stent framework in an oxygen or oxygen/nitrogen atmosphere, immersing in a mixture of hydrofluoric and perchloric acids, immersing in a potassium hydroxide solution and passing a current through the solution, or any of the known vacuum-deposition techniques such as plasma etching, or chemical vapor deposition. Using any of these processes, the thickness of the oxide zone can be controlled, to some extent, by altering the time and temperature of the oxidation process. Although there is some control over the porosity, including the size and number of pores, the strength of the oxide zone suffers as porosity increases. This is especially detrimental for an oxide coatings on the surface of a stent. The stent must be crimped to a catheter or balloon during delivery, then expanded at the treatment site. The expansion and contraction of the diameter of the stent often causes the oxide coating to buckle and break from the stent surface, limiting the practical applications of these coatings.

Metals such as iron (Fe), cobalt (Co) and copper (Cu) form multivalent cations, and therefore, are oxidized to multiple oxidation products. For example, upon exposure to oxygen (O₂), Fe is oxidized in stepwise fashion first to FeO, next to Fe₃O₄, and finally to Fe₂O₃. Thus, when the surface of a metal containing Fe is exposed to oxidizing conditions at high temperature, first a zone of FeO forms on the surface of the metal. Next, the FeO on the surface of the oxide zone, where the partial pressure of O₂ is highest, is further oxidized to Fe₃O₄. Since the oxide zone is porous, O₂ penetrates to the metal/oxide interface, and the FeO zone continues to form at the surface of the metal. Similarly, the Fe₃O₄ on the outer surface of the oxide zone undergoes a further oxidation step to Fe₂O₃, the highest oxidation state of Fe, while the two inner zones of FeO and Fe₃O₄ continue to form. The result, as shown in FIG. 1 a mixed metal, metal oxide system 100. Fe oxide coating 102 on the surface of Fe-containing metal 104, comprises FeO zone 106 at the metal/oxide interface, Fe₃O₄ zone 108 overlaying FeO zone 106, and external Fe₂O₃ zone 110. The formation rate of each oxide, and therefore the thickness of each zone can be regulated by the temperature of the metal during oxidation.

Oxidation of metals can also be carried out at elevated temperatures in an atmosphere of gaseous carbon dioxide (CO₂) or sulfur dioxide (SO₂). For example, at the metal surface, CO₂ reacts with the metal to form carbon monoxide (CO) and the metal oxide. In addition to the metal oxidation reaction, the carbon may either precipitate at the metal/oxide interface or react with the metal to form metal carbide. Similarly, metal oxidation in the presence of SO₂ forms metal oxide, metal sulfide and/or sulfide precipitate. In the case, of either reactant, the properties of the oxide zone are altered by the metal carbide or metal sulfide content.

Metal oxides are crystalline structures, and the porosity of a metal oxide coating is determined largely by the size of the component crystals. Oxidation is initiated at nucleation sites on the surface of the metal. The number and density of nucleation sites depends on the structure of the metal surface. The density of nucleation sites can be reduced by cold working, annealing or melting the metal surface. Similarly, the density of nucleation sites can be increased by etching the surface of the metal. Metal carbide and metal sulfide molecules formed at the metal/metal oxide interface migrate through the metal oxide zone and provide additional nucleation sites away from the metal/metal oxide interface.

Some metal oxides molecules are volatile at elevated temperatures and as these molecules volatilize, the porosity of the zone increases. Therefore, the porosity of a metal oxide zone can be modified by changing the temperature to first form one or more metal oxides, and then to volatilize some of the metal oxide molecules.

It would be desirable, to provide an implantable therapeutic agent eluting stent having a porous mixed metal oxide, and metal carbide or metal sulfide coating of optimal thickness and porosity that exhibits minimal chipping and flaking of the metallic coating when the stent is contracted or expanded during delivery and deployment. Such a stent would overcome many of the limitations and disadvantages inherent in the devices described above.

### SUMMARY OF THE INVENTION

One aspect of the present invention provides a system for treating abnormalities of the cardiovascular system comprising a catheter and a therapeutic agent-carrying stent disposed on the catheter. The stent includes a metallic stent framework having a porous therapeutic agent carrying zone formed on at least a portion of the surface of the stent framework. The porous therapeutic agent carrying zone comprises oxidation and reduction products of the stent framework.

Another aspect of the invention provides a stent comprising a metallic stent framework having a porous therapeutic agent carrying zone formed on at least a portion of the surface of the metallic stent framework. The porous therapeutic agent carrying zone includes oxidation and reduction products of the metallic stent framework.

Another aspect of the invention provides a method for manufacturing a therapeutic agent carrying stent comprising, first, selecting a desired porosity and thickness of a therapeutic agent carrying zone that will overlay the stent framework. The method further comprises determining a controlled environment based on the selected porosity and thickness of the therapeutic agent carrying zone, and exposing the metallic stent framework to the controlled environment. The method further comprises oxidizing at least a portion of the stent framework and reducing another portion of the stent framework within the controlled environment, and finally, forming the drug carrying zone having the desired porosity and thickness as a result of the oxidation and reduction reactions.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of an oxide coating having three zones on the surface of a metal or metal alloy containing Fe;

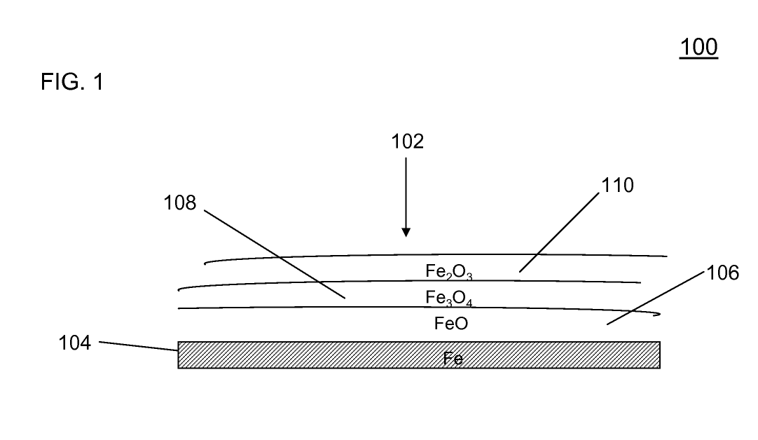

FIG. 2 is a schematic illustration of a system for treating a vascular condition including a therapeutic agent carrying stent coupled to a catheter, in accordance with one embodiment of the present invention;

FIG. 3 is a schematic illustration of the formation of a mixed metal oxide, metal carbide therapeutic agent carrying zone on the surface of a metal stent framework, in accordance with the present invention;

FIG. 4 is a schematic illustration of the formation of a mixed metal oxide, metal sulfide therapeutic agent carrying zone on the surface of a metal stent framework, in accordance with the present invention; and

FIG. 5 is a flow diagram for manufacturing a therapeutic agent carrying stent having a coating comprising oxidation and reduction products of the metal in the stent framework.

### DETAILED DESCRIPTION

Throughout this specification, like numbers refer to like structures.

The present invention is directed to a system for treating abnormalities of the cardiovascular system comprising a catheter and a therapeutic agent-carrying stent disposed on the catheter. A porous zone is formed at the surface of the stent by exposing a metallic stent framework to a reaction environment in which some metal atoms on the surface of the stent framework are oxidized to a metal oxide and other atoms are reduced to metal carbide or metal sulfide.

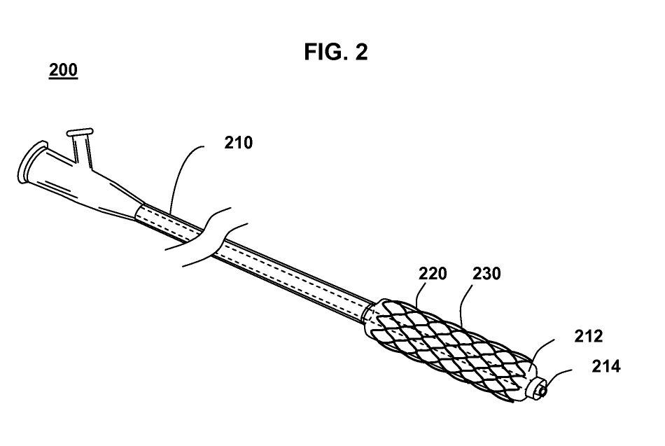

FIG. 2 shows an illustration of a system 200 for treating a vascular condition, comprising therapeutic agent carrying stent 220 coupled to catheter 210, in accordance with one embodiment of the present invention.

In an exemplary embodiment of the present invention, catheter 210 includes a balloon 212 that expands and deploys therapeutic agent carrying stent 220 within a vessel of the body. After positioning therapeutic agent carrying stent 220 within the vessel with the assistance of a guide wire traversing through guide wire lumen 214 inside catheter 210, balloon 212 is inflated by pressurizing a fluid such as a contrast fluid or saline solution that fills a tube inside catheter 210 and balloon 212. Therapeutic agent carrying stent 220 is expanded until a desired diameter is reached; then the contrast fluid is depressurized or pumped out, separating balloon 212 from therapeutic agent carrying stent 220 and leaving the therapeutic agent carrying stent 220 deployed in the vessel of the body. Alternately, catheter 210 may include a sheath that retracts to allow expansion of a self-expanding version of therapeutic agent carrying stent 220. Therapeutic agent carrying stent 220 includes a stent framework 230. In one embodiment of the invention, a porous zone is formed at the surface of at least a portion of metallic stent framework 230.

In one embodiment of the invention, the stent framework comprises one or more of a variety of biocompatible metals such as stainless steel, titanium, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys. The metal composition gives the stent framework the mechanical strength to support the lumen wall of the vessel, sufficient longitudinal flexibility so that it can be transported through the cardiovascular system, and provides a metallic substrate for the oxidation and reduction reactions that produce a porous coating.

The stent framework is formed by shaping a metallic wire or laser cutting the stent from a metallic sheet, or any other appropriate method. If needed, the surface of the stent framework is cleaned by washing with surfactants to remove oils, mechanical polishing, electropolishing, etching with acid or base, or any other effective means to expose a uniform metal surface.

The metallic surface of the stent framework is the substrate of the oxidation reaction, and as such, provides nucleation sites that initiate oxide formation, and becomes the interface between the metal reactant and the oxide zone. The number and distribution of the available nucleation sites for the oxide forming reaction depend on the crystalline structure of the metal. One or more metal oxides produced by the oxidation reaction also have crystalline structures that are initiated at the nucleation sites, and then grow to crystals. The size and shape of the oxide crystals depend on the length of time of the oxidation reaction and the charge on the activated metal ion, respectively. Therefore, in one embodiment of the invention, the crystalline structure of the metallic surface of the stent framework is modified to provide a desired number and distribution of nucleation sites on the metallic surface by processes such as cold working, annealing, and melting.

The rate of oxidation-reduction reactions is characteristic of each metal, and is highly temperature dependent. Although most metals oxidize slowly at room temperature, oxidation proceeds rapidly above 500 C. For example, Chromium (Cr) is rapidly oxidized to Cr₂O₃ at temperatures above 950 C. Similarly, Fe oxidizes rapidly at temperatures above 570 C. Consequently, if heated to a temperature above 950 C, a metal alloy containing Fe and Cr would produce a mixed oxide zone comprising Fe oxide (including FeO, Fe₃O₄, and Fe₂O₃) and Cr₂O₃. In contrast, if heated to 570 C, the same Fe/Cr metal alloy would produce a mixed oxide zone comprising predominantly Fe oxide (FeO, Fe₃O₄, and Fe₂O₃) with relatively low Cr₂O₃ content. In one embodiment of the invention, the temperature of the oxidation reduction reaction is selected, based on the composition of the metallic stent framework, to produce a metal oxide zone having the desired composition of oxides derived from each component metal in the stent framework. The useful temperature range is, however, limited to temperatures below the melting point of the metal or alloy. In one embodiment of the invention, the reaction temperature(s) are between about 500 C and 0.8 of the melting temperature of the metal or metal alloy comprising the region of the stent framework undergoing oxidation reduction reactions.

When metals are subjected to a CO₂ or SO₂ atmosphere at elevated temperature, a series of chemical reactions take place. Initially, the metal is oxidized and the oxidizing agent, either CO₂ or SO₂ is reduced to carbon monoxide (CO) or sulfur monoxide (SO), respectively. But as the oxidation reaction proceeds, and the thickness of the metal oxide zone increases, equilibria among the various chemical species are established. For example, if CO₂ is the oxidant and M represents the metal, the equilibria among the chemically reactive species in the growing oxide zone include:

CO₂ [symbol] CO+O²⁻

M [symbol] MO

2CO [symbol] CO₂+C

M+C [symbol] MC

where C is an activated carbon atom. If the metal is capable of forming stable carbides (MC), these compounds may also be present in the oxide zone, making it a mixed metal oxide, metal carbide zone. In one embodiment of the invention, the therapeutic agent carrying zone of a metallic stent framework is a mixed metal oxide, metal carbide zone.

An analogous set of reaction equilibria describe the reactants and products under conditions of high temperature in the presence of SO₂. Thus, if the metal, M, is capable of forming stable sulfides (MS) these compounds may be distributed throughout the metal oxide zone, making it a mixed metal oxide, metal sulfide zone. In one embodiment of the invention, the therapeutic agent carrying zone of a metallic stent framework is a mixed metal oxide, metal sulfide zone.

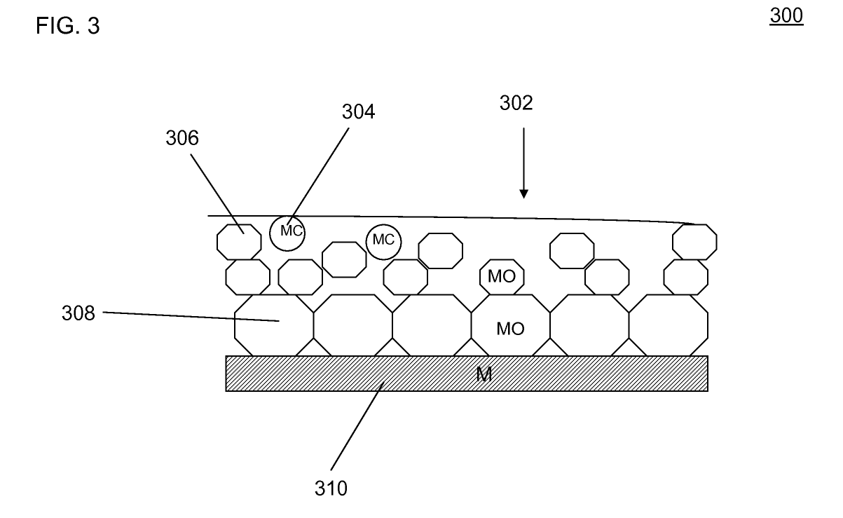

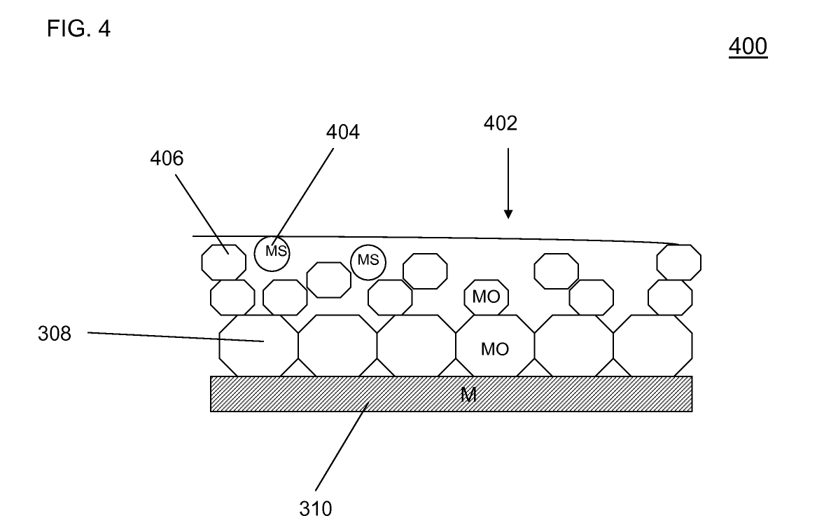

Metal carbide or metal sulfide molecules distributed throughout the crystalline metal oxide zone provide nucleation sites for the formation of metal oxide crystals in addition to those nucleation sites on the metal surface at the metal/metal oxide interface. As shown in FIG. 3, additional metal carbide nucleation sites 304 alter metal oxide zone 302 by initiating the formation of new metal oxide crystals 306 within the zone. Newly forming crystals 306 are smaller in size than metal oxide crystals 308 that were initiated at the interface between the surface of metallic stent framework 310 and metal oxide zone 302. FIG. 4 shows a similar process of metal oxide formation in the presence of SO₂. Metal on the surface of stent framework 310 is first oxidized to metal oxide crystals 308 at nucleation sites on the surface of metal stent framework 310. However metal sulfide ions 404 migrate into metal oxide zone 402, and become nucleation sites for newly forming metal oxide crystals 406 away from the surface of metal 310.

The rates of oxidation and reduction reactions of metals can be modified by using one or more catalysts. Catalysts such as magnesium chloride or silver sulfide can also provide nucleation sites for metal oxidation, or allow the reaction to proceed at a lower temperature than would be required in the absence of the catalyst. In one embodiment of the invention, catalysts such as magnesium chloride or silver sulfide are added to the reaction environment to modify the composition of the resultant mixed metal oxide, metal carbide or metal sulfide zone.

The porosity of a metal oxide coating generally increases as the metal oxide crystals increase in size and the coating coarsens, often resulting in poor retention of the therapeutic agent(s) to be delivered. In contrast, a mixed metal oxide, metal carbide or metal sulfide zone has more nucleation sites distributed throughout the zone, and therefore more, smaller crystals distributed throughout the zone. In one embodiment of the invention, as the therapeutic agent carrying zone increases in thickness, the porosity remains nearly constant due to the additional metal carbide or metal sulfide nucleation sites distributed throughout the therapeutic agent carrying zone. In contrast to a simple metal oxide zone, it is possible to attain a therapeutic agent carrying zone of any desired thickness while maintaining an optimal pore size for controlled release of the therapeutic agent.

A stent framework having the strength and flexibility needed may not have a metal composition that will produce a therapeutic agent carrying zone having the optimal porosity and thickness. In one embodiment of the invention, this problem is solved by coating the surface of the stent framework with a metal that will form a mixed metal oxide, metal carbide or metal sulfide zone having the desired porosity. This pore forming surface metal zone may be applied to the stent framework by electroplating, for example.

In one embodiment of the invention, the therapeutic agent carrying zone is discontinuous, leaving small fissures or cracks between various regions of the zone. The purpose of the fissures or cracks is to prevent the therapeutic agent carrying zone from buckling when the stent is expanded or contracted. In one embodiment of the invention the metal surface is an alloy, and the temperature during the oxidation reduction reaction of the metal is selected so that at least one of the metals in the alloy does not react, leaving gaps in the zone at the metallic surface. In another embodiment, the metal oxide, metal carbide or metal sulfide zone is made discontinuous by intermittently starting and stopping the reactions by raising and lowering the temperature in the reaction chamber. In yet another embodiment, an induction current, laser source, radio frequency, ultrasound infrared, electron beam, or other device is used to raise the temperature of a targeted area of the stent framework to a first temperature so that the oxidation reduction reactions take place in a highly localized region. The heat source is then moved to adjacent regions of the stent surface, so that various regions of the metallic surface are reacted separately, resulting in a discontinuous zone, which may have different compositions of metal oxides and metal carbides or metal sulfides depending on the composition of the metal surface, especially if it is an alloy. Alternatively, each region of a metal alloy surface may be reacted at a different temperature so that different metals react and the composition of each region of the discontinuous zone comprises different metal oxides, metal carbides and metal sulfides.

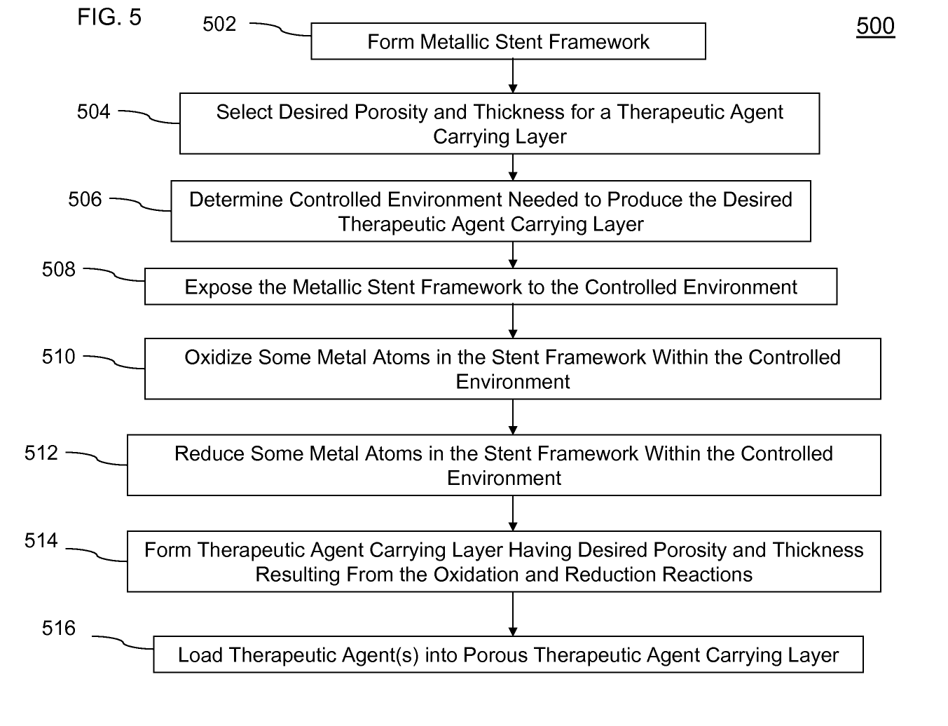

FIG. 5 is a flowchart of method 500 for manufacturing a therapeutic agent eluting stent in accordance with the present invention. The method includes forming a metallic stent framework, as indicated in Block 502. In some embodiments, a metallic wire is formed into a tubular shape about a mandrel. Alternatively, a sheet of metallic or polymeric material is laser cut and rolled into a tubular shape to form the stent framework. Using either method, a tubular stent framework is formed having a manufactured diameter that is intermediate between the diameter of stent framework in the compressed and the expanded configurations.

Next, the thickness and porosity of the therapeutic agent carrying zone are selected, as shown in Block 504. Targeted porosity will depend on the molecular weight and polarity of the therapeutic agent to be delivered; the optimal thickness of the zone will be determined by the amount of therapeutic agent to be delivered, and the period of time over which delivery is to take place.

In one embodiment of the invention, a controlled environment is selected (Block 506) that will produce a therapeutic agent carrying zone having the desired porosity and thickness. The controlled environment will comprise reaction conditions including an atmosphere comprising gaseous oxidizing and reducing agents such as CO₂ or SO₂, at an optimal partial pressure, elevated temperature, and time of exposure. In one embodiment, one or more catalysts are used during the course of the reaction.

Next, as indicated in Block 508, the portion of the stent framework to be oxidized and reduced is exposed to the controlled environment for the selected time of exposure. During exposure to the controlled environment, some metal atoms on the surface of the stent framework are oxidized to metal oxide (Block 510), and other metal atoms are reduced to either metal carbide or metal sulfide (Block 512), depending on whether the gaseous oxidizing/reducing agent is CO₂ or SO₂. The products of the oxidizing and reducing reactions form the therapeutic agent carrying zone on the surface of the stent framework having a porosity and thickness consistent with the desired characteristics for the therapeutic agent carrying zone (514). In one embodiment of the invention, the temperature is modified during the reaction to make the therapeutic agent carrying zone discontinuous. This is accomplished by raising and lowering the temperature to start and stop the reactions, or changing the temperature of different regions of the stent framework surface independently of the surrounding regions. In another embodiment, the temperature is modified to volatilize one or more of the metal oxides and thereby adjust the porosity of the therapeutic agent carrying zone.

Finally, in one embodiment of the invention, the pores of the therapeutic agent carrying zone are filled with one or more therapeutic agents, such as anticoagulants, antiinflammatories, fibrinolytics, antiproliferatives, antibiotics, therapeutic proteins or peptides, recombinant DNA products, or other bioactive agents, diagnostic agents, radioactive isotopes, or radiopaque substances are applied to the therapeutic agent-carrying zone of the stent in a formulation appropriate for the therapeutic agent(s) to be delivered (Block 416). The formulation containing the therapeutic agent may additionally contain excipients including solvents or other solubilizers, stabilizers, suspending agents, antioxidants, and preservatives, as needed to deliver an effective dose of the therapeutic agent to the treatment site. In some embodiments of the invention, the formulation is applied as a liquid to the therapeutic agent-carrying zone of the stent framework so that the porous structures are filled with the formulation. The formulation is then dried to remove the solvent using air, vacuum, or heat, and any other effective means of causing the formulation to adhere to the stent framework.

The completed stent may then be compressed and mounted on a catheter, expanded at the delivery site, and otherwise handled as needed with minimal chipping, flaking, and loss of the therapeutic agent or the mixed metal oxide, metal carbide or metal sulfide coating.

While the invention has been described with reference to particular embodiments, it will be understood by one skilled in the art that variations and modifications may be made in form and detail without departing from the spirit and scope of the invention.

1. A system for treating a vascular condition comprising:
a catheter;
a stent disposed on the catheter, the stent comprising a metallic stent framework and a porous therapeutic agent carrying zone formed within at least a portion of a surface of the metallic stent framework, wherein the porous therapeutic agent carrying zone includes at least one targeted controlled environment oxidation product and at least one targeted controlled environment reduction product of the metallic stent framework.

2. The system of claim 1 wherein the metallic stent framework comprises at least one metal selected from the group consisting of iron, magnesium, aluminum, titanium, cobalt, chromium, nickel, platinum, iridium, gold, chromium/cobalt alloys, cobalt/titanium alloys, nickel/titanium alloys, platinum/tungsten alloys, chromium/nickel alloys, stainless steel, and other medically acceptable metals.

3. The system of claim 1 wherein the targeted controlled environment oxidation products and targeted controlled environment reduction products of the metallic stent framework include at least one metal oxide and at least one metal carbide.

4. The system of claim 3 wherein a porous structure of the porous therapeutic agent carrying zone is determined by the quantity and distribution of the metal carbide.

5. The system of claim 1 wherein the targeted controlled environment oxidation products and targeted controlled environment reduction products of the metallic stent framework include at least one metal oxide and at least one metal sulfide.

6. The system of claim 5 wherein a porous structure of the porous therapeutic agent carrying zone is determined by the quantity and distribution of the metal sulfide.

7. The system of claim 1 further comprising at least one therapeutic agent releasably disposed within the porous therapeutic agent carrying zone.

8. A stent comprising a metallic stent framework and a porous therapeutic agent carrying zone formed on at least a portion of a surface of the metallic stent framework, wherein the porous therapeutic agent carrying zone includes at least one targeted controlled environment oxidation product and at least one targeted controlled environment reduction product of the metallic stent framework.

9. The stent of claim 8 wherein the stent framework comprises at least one metal selected from the group consisting of iron, magnesium, aluminum titanium, cobalt, chromium, nickel, platinum, iridium, chromium/cobalt alloys, cobalt/titanium alloys, chromium/nickel alloys, stainless steel, and other medically acceptable metals.

10. The stent of claim 8 wherein the targeted controlled environment oxidation products and targeted controlled environment reduction products of the metallic stent framework include at least one metal oxide and at least one metal carbide.

11. The stent of claim 10 wherein a porous structure of the porous therapeutic agent carrying zone is determined by the quantity and distribution of the metal carbide in the therapeutic agent carrying zone.

12. The stent of claim 8 wherein the oxidation and reduction products of the metallic stent framework include at least one metal oxide and at least one metal sulfide zone.

13. The stent of claim 12 wherein a porous structure of the porous therapeutic agent carrying zone is determined by the quantity and distribution of the metal sulfide in the therapeutic agent carrying zone.

14. The stent of claim 8 further comprising a one or more therapeutic agents disposed in the porous metallic zone.

15. A method of manufacturing a therapeutic agent-carrying stent comprising:
selecting a desired porosity and thickness of a therapeutic agent carrying zone of a stent framework;
determining a controlled environment based on the selected porosity and thickness of the therapeutic agent carrying zone;
exposing the metallic stent framework to the determined controlled environment;
oxidizing at least a portion of the stent framework within the controlled environment;
reducing at least a portion of the stent framework within the controlled environment;
forming the therapeutic agent carrying zone including a porosity and thickness consistent with the selected porosity and thickness based on the oxidation and reduction reactions.

16. The method of claim 15 further comprising heating at least a portion of the stent framework to a temperature between about 500 C and about 0.8 times the melting temperature of the metal comprising the stent.

17. The method of claim 16 wherein a heat source is selected from the group consisting of induction current, laser, radio frequency, ultrasound, infrared, and electron beam irradiation

18. The method of claim 15 further comprising exposing the stent framework to a gaseous atmosphere comprising non-atmospheric levels of carbon dioxide and forming a porous metallic zone comprising metal oxide and metal carbide.

19. The method of claim 15 further comprising exposing the stent framework to a gaseous atmosphere comprising non-atmospheric levels of sulphur dioxide and forming a porous metallic zone comprising metal oxide and metal sulfide.

20. The method of claim 15 further comprising altering at least one surface characteristic of the surface of the stent framework by one or more procedures selected from the group consisting of cold working, annealing and melting the surface metal.

21. The method of claim 15 further comprising applying a pore formation metal to the surface of the stent framework.

22. The method of claim 15 further comprising disposing one or more therapeutic agents within the porous therapeutic agent carrying zone.
